(19) United States
(12) Patent Application Publication
(10) Pub. No.: US 2004/0152071 A1
(43) Pub. Date: Aug. 5, 2004

(54) Targeted delivery of drugs for the treatment of viral infections

(76) Inventor: W. Page Faulk, Indianapolis, IN (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/477,997

(22) PCT Filed: May 15, 2002
(86) PCT No.: PCT/US02/11892

(51) Int. Cl. A01N 37/18; C12Q 1/70; A61K 38/16; A61K 38/00

(52) U.S. Cl. 435/5; 514/2

(57) ABSTRACT

Conjugates of transferrin or transcobalamin with anti-viral agents are useful in the treatment of viral infections. Suitable anti-viral agents include apoptosis inducing compounds, compounds which inhibit the replication of the virus, a cytotoxic antibiotic, an alkylating agent, a plant toxin, and a bacterial mutant toxin. Transferrin or transcobalamin is preferably coupled to the anti-viral agent by means of glutaraldehyde.

### FIELD OF THE INVENTION

This invention relates generally to the field of bioaffecting materials and, more specifically to bioaffecting materials suitable for treating cells, including human cells, that are stressed, especially those stressed as a result of a viral infection.

### BACKGROUND OF THE INVENTION

Two common problems in treatments which involve drugs are drug-toxicity, which debilitates patients, and drug-resistance, which requires more drugs and thus amplifies the problem of drug-toxicity, often resulting in death. One way to solve the problem of drug-toxicity is to deliver drugs so they are targeted only to the diseased cells. Many researchers are working to develop antibodies to deliver drugs, and this approach holds promise, but antibodies are not without problems. For example, they often cross-react with normal tissues, and they can damage blood vessels (e.g., vascular leak syndrome) and cause dangerous allergic reactions (e.g. anaphylaxis).

The treatment of malignant cells by the delivery of drugs, including drugs that are toxic to such cells, is not new. U.S. Pat. Nos. 4,886,780; 4,895,714; 5,000,935; and 5,108,987 to Faulk and U.S. Pat. No. 4,590,001 to Stjernholm et. al., describe cytotoxic or radioimaging materials conjugated to proteins, mainly to transferrin, as treatments for cancerous cells or for imaging cancerous cells. These publications disclose useful methods for making and using such materials.

It is known that stressed cells, such as, for example, human cells hosting a viral infection and human cells invaded by cancer, call for an increased delivery of nutrients, such as iron, by presenting an increased number of receptors for nutrient carriers, such as transferrin in the case of iron. The increase in receptors for nutrient carriers in stressed cells is known to be relatively constant and orders of magnitude greater in number than in unstressed cells, which are known to show receptors intermittently and in relatively smaller numbers. The publications listed above, and others, disclose taking advantage of the increased number of receptors, especially for transferrin, presented by cancer containing cells to deliver imaging materials or drugs or both to the stressed cell.

No single study has asked if all stressed cells have up-regulated transferrin receptors, or if all normal cells have down-regulated transferrin receptors, but data from many quarters suggest that all normal cells have down-regulated transferrin receptors. For example, immature erythrocytes (i.e., normoblasts and reticulocytes) have transferrin receptors on their surfaces, but mature erythrocytes do not (Lesley J, Hyman R, Schulte R and Trotter J. Expression of transferrin receptor on murine hematopoietic progenitors. Cell Immunol 1984; 83: 14-25). Circulating monocytes also do not have up-regulated transferrin receptors (Testa U, Pelosi E and Peschle C. The transferrin receptor. Crit Rev Oncogen 1993; 4: 241-276), and macrophages, including Kupffer cells, acquire most of their iron by a transferrin-independent method of erythrophagocytosis (Bothwell T A, Charlton R W, Cook J D and Finch C A. Iron Metabolism in Man, Blackwell Scientific, Oxford, 1979). In fact, in vivo studies indicate that virtually no iron enters the reticuloendothelial system from plasma transferrin (for review, see Ponka P and Lok C N. The transferrin receptor: role in health and disease. Int J Biochem Cell Biol 1999; 31: 1111-1137.). Macrophage transferrin receptors are down-regulated by cytokines such as gamma interferon (Hamilton T A, Gray P W and Adams D O. Expression of the transferrin receptor on murine peritoneal macrophages is modulated by in vitro treatment with interferon gamma. Cell Immunol 1984; 89: 478-488.), presumably as a mechanism of iron-restriction to kill intracellular parasites (Byrd T F and Horowitz M A. Interferon gamma-activated human monocytes downregulate transferrin receptors and inhibits the intracellular multiplication of Legionella. pneumophila by limiting the availability of iron. J Clin Invest 1989; 83: 1457-1465.).

In resting lymphocytes, not only are transferrin receptors down-regulated, but the gene for the transferrin receptor is not measurable (Kronke M, Leonard W, Depper J M and Greene W C. Sequential expression of genes involved in human T lymphocyte growth and differentiation. J Exp Med 1985; 161: 1593-1598). In contrast, stimulated lymphocytes up-regulate transferrin receptors in late G₁ (Galbraith R M and Galbraith G M. Expression of transferrin receptors on mitogen-stimulated human peripheral blood lymphocytes: relation to cellular activation and related metabolic events. Immunology 1983; 133: 703-710). Receptor expression occurs subsequent to expression of the c-myc proto-oncogene and following up-regulation of 1L-2 receptor (Neckers L M and Cossman J. Transferrin receptor induction in mitogen-stimulated human T lymphocytes is required for DNA synthesis and cell division and is regulated by interleukin 2. Proc Nat Acad Sci USA 1983; 80: 3494-3498.), and is accompanied by a measurable increase in iron-regulatory protein binding activity (Testa U, Kuhn L, Petrini M, Quaranta M T, Pelosi E and Peschle C. Differential regulation of iron regulatory element-binding protein(s) in cell extracts of activated lymphocytes versus monocytes-macrophages. J Biol Chem 1991; 266: 3925-3930), which stabilizes transferrin receptor mRNA (Seiser C, Texieira S and Kuhn L C. Interleukin-2-dependent transcriptional and post-transcriptional regulation of transferrin receptor mRNA. J Biol Chem 1993; 268: 13,074-13,080.). This is true for both T and B lymphocytes (Neckers L M, Yenokida G and James S P. The role of the transferrin receptor in human B lymphocyte activation. J Immunol 1984; 133: 2437-2441), and is an IL-2-dependent response (Neckers L M and Trepel J B. Transferrin receptor expression and the control of cell growth. Cancer Invest 1986; 4: 461-470).

The best understood material mentioned in the above-listed publications is a conjugate of transferrin and doxorubicin, a well known and effective cytotoxic molecule. Although effective against cancers, doxorubicin has a maximum lifetime dosage for humans due to its cumulative cardiotoxicity. The conjugate has been shown to be effective in surprisingly low doses in killing a variety of types of cancers, including drug-resistant cancers in humans.

Well known and presently used methods for treating serious viral infections, such as infections by the human immunodeficiency virus (HIV) include blocking cell receptors that the virus uses to enter the cell, interfering with fusion mechanisms, and interfering with cell enzymes hijacked by the virus such as proteases and reverse transcriptases. Such methods and the drugs used in the methods, although effective in prolonging the life of seriously ill patients, have not resulted in wholesale cures. The materials themselves are well known to be prohibitively expensive for use in many parts of the world. Often, treatment with such drugs burdens a patient with complex dosing schemes and presents unpleasant side effects.

There is a need for materials for use in the treatment of high profile conditions such as AIDS, caused by HIV infections, that provide more effective results at a lower cost and fewer side effects for patients. There is also a need for materials for use in treating cells infected with a variety of other viral infections that burden societies, such as cytomegalovirus, adenoviruses, hepatitis viruses, herpes simplex viruses, and the like. There is also a need for drugs that kill such viruses and a variety of cancers without the use of cytotoxic materials even in small amounts.

### DESCRIPTION OF THE RELATED ART

All drugs currently approved by the FDA for treatment of AIDS patients are designed to attack specific stages in the life cycle of HIV in T lymphocytes (De Clercq, Clin Microbiol Rev 1995; 8:200). In light of this, there currently are only two pharmacological strategies to win the war on AIDS. One of these is the use of reverse transcriptase inhibitors (both nucleoside and non-nucleoside) to block viral replication during the RNA reverse transcription to DNA (Cratlin et al., Virology 1998;244:87). All of the nucleoside reverse transcriptase inhibitors are prodrugs that require intracellular metabolism to active triphosphate analogues (Lavie et al, Nature Med 1997; 3:922), and their use often results in drug resistance (Hazuda & Kuo, Nature Med 1997; 3:836). The other strategy involves inhibitors of HIV proteases responsible for processing of the gag and gag-pol polyproteins during virion maturation (Gulnik et al., Vit & Hormones. 2000; 58:213), but treatment with protease inhibitors also often results in drug resistance (Olsen et al., J Biol Chem 1999; 274:23699). The development of drug resistance is a major problem with antiretroviral drugs (Calvez, Antiviral Therapy 1998; 3(Suppl 4): 5).

In addition to classical drug designs based on the life cycle of the virus, there is another approach that is based on the immunobiology of T lymphocytes, which are the cells in AIDS patients that host the virus (Ho et al., Nature 1995; 373:123). Two recent studies suggest this is a promising approach. First, HIV-primed CD8 T lymphocytes have been shown to diminish viremia when transferred into AIDS patients (Drodic et al., Nature Med 1999; 5:34). Second, a caspase-3 proenzyme with an HIV protease activation site has been shown to activate apoptosis when transfected into HIV-infected T lymphocytes but not when transfected into uninfected cells (Vocero-Akbani et al., Nature Med 1995; 5:29). These reports suggest the possibility of controlling HIV infections by selectively killing the cells in which the virus lives.

An established observation about AIDS patients is that the immunodeficiency component of their disease is due to a deficiency of CD4 lymphocytes, which are the subpopulation of T lymphocytes in which HIV lives (Office of AIDS Research, Ann Intern Med 1998; 128:1057). The reason these cells are deficient in AIDS patients is that the virus initiates an enzymatically-mediated process of programmed cell death, which eliminates the cells by a reaction known as apoptosis (Cicala et al., Proc Natl Acad Sci USA 2000; 97:1178). When infected cells are killed by the virus, viremia increases, as measured by plasma HIV RNA levels (Report of NIH Panel, Ann Inern Med 1998; 128 (No. 12, pt 2): 1057), but when infected cells are killed by antiretroviral drugs, viremia decreases (Katzenstein et al., N Engl J Med 1996; 335:1091), and decreasing viremia is associated with improved clinical outcome (O'Brien et al., N Engl J Med 1996; 334:426).

Kast, et al., U.S. Pat. No. 6,242,176, discloses a method of delivering a pharmacologically active substance to a papillomavirus-infected cell. This method uses a complex including a pharmacologically active substance and a ligand recognizing CD16. The complex is exposed to the papillomavirus-infected cells under conditions sufficient for the ligand to bind CD16 on the cells. The pharmacologically active substance is thus brought into proximity to, and delivered to, the infected cell. The use of this method permits the targeted delivery of the pharmacologically active substance to the infected cells. This permits the employment of relatively high concentrations of many pharmacologically active agents to be delivered to the infected cells without many of the concomitant side effects attributed to the activity of such agents in noninfected cells.

The targeted delivery of drugs has the advantage of increasing efficacy while using less drug, thereby decreasing toxicity and causing less damage to normal cells, all of which effectively decrease costs and increase the quality of patient care. Targeted delivery also avoids drug-resistance, which is activated by the non-specific entrance of drugs into cells (Marbeuf-Gueye C, Ettori D, Priebe W, Kozlowski H and Garnier-Suillerot A. Correlation between the kinetics of anthracycline uptake and the resistance factor in cancer cells expressing the multidrug resistance protein or the P-glycoprotein. Biochem Biophy Acta 1999; 1450: 374-384). Because transferrin-drug conjugates enter cells specifically by employing a receptor-specific pathway (Klausner RD, vanReuswoude J, Ashwell G, Kempf C, Schechter A N, Dean A and Bridges K. Receptor-mediated endocytosis of transferrin in K562 cells. J Biol Chem 1983; 258: 4715-4724; Berczi A, Ruthner M, Szuts V, Fritzer M, Schweinzer E and Goldenberg H. Influence of conjugation of doxorubicin to transferrin on the iron uptake by K562 cells via receptor-mediated endocytosis. Euro J Biochem 1993; 213: 427-436.), they are trafficked around drug-resistance mechanisms, such as efflux pumps in resistant cells.

### SUMMARY OF THE INVENTION

The present invention provides a material for treating viral infections such as HIV, cytomegalovirus, adenovirus, hepatitis virus, and herpes simplex virus infections. The material comprises an infected cell targeting agent that binds with receptors up regulated by virally infected cells, such as transferrin or transcobalamin, conjugated with a component that inhibits the conjugate from leaving the cell after binding of the targeting agent with a receptor. The material has been found to induce apoptosis in cells infected with a virus. Suitable drugs include but are not limited to doxorubicin and methotrexate. The present invention also provides a method for treating patients infected with a virus and a composition containing the conjugate.

### BRIEF DESCRIPTION OF THE DRAWINGS

FIG. 1 shows the dose-response curve obtained for the inhibition of the ROJO strain of HIV-1 virus living in human blood cells by a transferrin-doxorubicin (TR-DOX) conjugate.

FIG. 2 shows the dose response curve obtained by exposing human liver cells infected with Hepatitis B virus (HBV) to increasing concentrations of a transferrin-doxorubicin (TR-DOX) conjugate.

FIG. 3 shows the effect of a transferrin-doxorubicin (TR-DOX) conjugate on Cytomegalovirus (CMV) living in human lung cells.

### DETAILED DESCRIPTION OF THE INVENTION

The above discussed needs are filled by a conjugate for treating cells, especially stressed cells, that in one embodiment includes a carrier that is attracted to a receptor that is expressed in higher numbers or more frequently by cells under stress, conjugated with a material that effectively prevents the conjugated material from leaving the cell following binding between the carrier and a receptor that has been up regulated to the cell surface.

The carrier can be any useful material that is attracted to receptors on cells that present in higher numbers or more frequently when a cell is stressed. Preferably, the carrier is transferrin.

The agent may be any agent that accomplishes the purpose of increasing the affinity of the carrier for the receptor. The mechanism by which the affinity is increased may vary and may include, for example, attachment of the agent to phospholipids on the cell surface or steric reconfiguration of the carrier, provided that the effect of the mechanism is to interfere with the normal displacement of the carrier from the receptor after contact. The best known such agent at the moment is doxorubicin. However, any useful material that accomplishes the desired effect may be used.

Attachment of the agent to the carrier may be by any mechanism that prevents their separation, at least until after the carrier has been positioned in the corresponding receptor with increased affinity. Presently, the best known mechanism for attachment for the transferrin-doxorubicin conjugate is a gluteraldehyde linker, but it could be any useful material for the carrier/agent combination in question.

The present invention is based on the surprising and unexpected discovery that the well known transferrin-doxorubicin material that had been believed to deliver doxorubicin to cells for killing of the cell by normal doxorubicin toxicity mechanisms, such as DNA intercalation, does not, in fact, result in the cell being killed by that mechanism. It has been found that the cells treated by contact with the conjugate experience apoptotic type cell death rather than by necrosis as is found when cancer cells are treated with doxorubicin alone.

A fundamental aspect of the present invention is based on the observation that viremia decreases as the number of infected CD4 T lymphocytes are removed from the circulation (Betts et al., AIDS Res Hum Retrovir 1999; 15:1219), and that patient well being is increased as viremia decreases (Wenfurt et al., Medical Care 2000; 38:404). In light of this, one mechanism of lowering the viral load is via HIV-1-specific cytolytic CD8 T lymphocytes (Lubaki et al., J. Acquir 1 mm Def Synd 1999; 22:19). Another mechanism to remove specific target cells is via the targeted delivery of protein conjugates of cytotoxic and/or antiretroviral drugs to cells that manifest a surface receptor for the carrier protein, as has been shown by using doxorubicin conjugates of human transferrin to kill human cancer cells that manifest transferrin receptors on their surfaces (Barabas et al., J Biol Chem 1992; 267:9437). Indeed, the binding reaction is so specific that doxorubicin conjugates of transferrin have been shown to bind isolated transferrin receptors even in the absence of cells (Ruthner et al., 1993; 54:35). In addition, the binding of transferrin-doxorubicin conjugates to transferrin receptors on cellular plasma membranes is more avid than the binding of unconjugated transferrin to transferrin receptors on cellular plasma membranes, probably because of additional interactions between the conjugate and constituents of the plasma membranes (Szuts et al., J Receptor Res 1993; 13:1041).

The present invention utilizes drug conjugates of human transferrin to be used in the targeted delivery of drugs to cells infected with viruses such as HIV. The primary reason that this invention is effective is that HIV-infected CD4 T lymphocytes up-regulate transferrin receptors to their surfaces (Ohno et al., Virolgy 1997; 238:305). However, there are several additional reasons why drug conjugates of transferrin are effective against HIV-infected T lymphocytes. First, many cytotoxic drugs, including doxorubicin (Ferraro et al., Cancer Res 2000; 60: 1901), induce apoptosis (Debatin, Tox Lett 2000; 112/113: 41), indicating that transferrin conjugates of doxorubicin are useful conjugates to induce apoptosis in cells with up-regulated transferrin receptors. Second, activation of transferrin receptors, by for instance transferrin-doxorubicin conjugates, is associated with drug-induced apoptosis (Leardi et al., Brit J Haematol 1998; 102:746), as well as with the control of calcium channels (Sainte-Marie et al., Eur J Biochem 1997; 250: 689-97) that are thought to be involved in effector pathways of apoptosis (Lepple-Wienhues et al., Proc Natl Acad Sci USA 1999; 96:13795; and Hueber, Nature Cell Biol 2000; 2: E23). Third, although transferrin receptors and apoptosis-inducing Fas receptors (APO-1/CD95) are located on the surface of cells (Findley & Zhou, Leukemia 1999; 13:147), when transferrin conjugates are delivered to cells with up-regulated transferrin receptors the conjugates are bound and eventually endocytosed (Berczi et al, Eur J Biochem 1993; 213:427), thus offering the possibility of killing by mechanisms activated at the cell surface as well as within the cell (Barabas et al., J Biol Chem 1992; 267:9437).

The immunobiology of purging T lymphocytes from the body is to initiate a pathway of programmed cell death by a process known as apoptosis (Pinkoski & Green, Cell Death & Dif 1999; 6: 1174-1181). This can occur in the thymus gland or in the peripheral circulation (Le Bon et al., Int Immunol 1999; 11: 373). Although there are several mechanisms of apoptosis, they all function to eliminate selected cells from the body (Martinez & Kraus, Int Rev Immunol 1999; 18: 527). The present invention is a novel way to eliminate HIV-infected T lymphocytes. The strategy of this approach is to eliminate the infected T lymphocytes by clonal deletion via apoptosis. This will be accomplished by using a drug-protein conjugate for the targeted delivery of cytotoxic and/or antiretroviral drugs to infected cells.

Targeted delivery of the conjugate of this invention results from the protein in the conjugate having a binding affinity for receptors that are up-regulated to the surface of HIV-infected lymphocytes (Ohno et al., Virology 1997; 238:305). In addition, the drug in the drug-transferrin conjugate can be methotrexate, which is known to cause apoptosis and clonal deletion of activated peripheral T lymphocytes (Genestier et al., J Clin Invest 1998; 102: 322). Other than being present on the surfaces of cancer cells (Yeh et al., Vox Sang 1.984; 46: 217-223), infected cells (Ohno et al., Virology 1997; 238: 305) and antigen-stimulated T lymphocytes (Bayer et al., J Leukoc Biol 1998; 64: 19), transferrin receptors usually are not present on the surface or normal, adult, resting cells (Berczi et al., Arch Biochem Biophy 1993; 300: 356). Thus, normal cells will not be affected, and the only cells to be eliminated by a methotrexate-transferrin conjugate will be the infected T lymphocytes.

The way to realize this invention of targeted drug delivery to HIV infected cells is to focus on the use of transferrin, which carries iron in the blood. Transferrin can be obtained by isolation from blood plasma, from commercial suppliers, or from recombinant technology (Ali et al., J Biol Chem 1999; 274: 24066). To form the drug protein conjugate, the transferrin molecule must be modified in such a way as to prepare it to be coupled with a cytostatic or an antiretroviral drug. The drug can be an apoptosis initiator such as methotrexate, a cytotoxic antibiotic such as doxorubicin or an alkylating agent, but any compound can be used, including plant toxins such a ricin, and bacterial mutant toxins such as modified diptheria toxin (Laske et al., Nature Med 1997; 41: 1039). Several coupling processes such as glutaraldehyde coupling (Berczi et al., Arch Biochem Biophys 1993; 300: 356), disulfide coupling (Sasaki et al., Jap J Can Res 1993; 84: 191) and benzoyl hydrazone coupling (Kratz et al., J Pharm Sci 1998; 87: 338) have been used to couple transferrin with other compounds. The wide variety of coupling procedures allows the conjugation of a broad range of cytotoxic drugs to transferrin, resulting in either permanent or dissociable bonding of drugs with the transferrin molecule. Following the coupling reaction, drug-protein conjugates can be separated from uncoupled drug and free protein, preferably by using chromatographic procedures.

Technical details of the conjugation procedure can vary, but the requirement of any procedure is to prepare defined conjugates that are (a) active in binding and killing experiments with viral infected cells, and that (b) do not bind or kill significant numbers of normal cells. In light of these requirements, the preferred method for preparing the conjugates according to the present invention is the following process:

The synthesis of large amounts of homogeneous transferrin-adriamycin conjugates with predetermined molecular ratios was done stoichiometrically by employing the only amino group of doxorubicin (DOX), which is at the 3′ amino position, to react with one of the two reactive groups on glutaraldehyde (GLU). Thus, the first step was drop-wise addition of a saline solution of DOX into a saline solution of GLU containing a solvent such as DMSO or another suitable cryopreservative, to a final concentration of a 1:1 molar ratio of DOX-to-GLU. The resulting solution of DOX-GLU was stirred three hours at room temperature in the dark.

The molarities of DOX and GLU were the same in the above reaction in order to produce a final solution of DOX-GLU that contains neither free DOX nor free GLU. However, there is the possibility of free GLU in solution if one GLU reacts with two DOX to produce DOX-GLU-DOX, but this possibility is minimized by the mass action kinetics generated by drop-wise addition of monovalent DOX into the solution of bivalent GLU. The volumes of these reactants are not restricted, so large amounts of homogeneous DOX-GLU can be prepared.

The second step in the conjugation reaction was drop-wise addition of DOX-GLU into a saline solution of transferrin (TRF). The TRF can be either iron-free (apo-transferrin) or iron-saturated (holo-transferrin). The desired molar ratio of DOX to TRF was obtained by appropriately adjusting the volume of TRF. The resulting solution of TRF-GLU-DOX was stirred for 20 hours at room temperature in the dark. Unlike the reaction of DOX with GLU, the reaction of DOX-GLU with TRF is not restricted to one binding site, for the GLU component of DOX-GLU can react with any one of several epsilon-amino lysine groups in the TRF molecule.

The number of DOX molecules bound to TRF was determined in the second step. For example, if the starting ratio of DOX-GLU to TRF was 7.2: 1.0, the final solution of TRF-GLU-DOX would have contained 2.5 molecules of DOX per molecule of TRF. However, if the starting ratio of DOX-GLU to TRF was 4.0:1.0, the final solution of TRF-GLU-DOX would have contained 1.4 molecules of DOX per molecule of TRF. Similarly, if the starting ratio of DOX-GLU to TRF was 2.5:1.0, the final solution of TRF-GLU-DOX would have contained 0.9 molecules of DOX per molecule of TRF. In this way, large amounts of TRF-GLU-DOX with predetermined ratios of DOX-to-TRF can be provided according to the need.

Further steps in the conjugation reaction were the addition of ethanolamine or another substance suitable for scavenging any excess linker, followed by centrifugation and dialysis. Although reactions with DOX and TRF theoretically consume all of the GLU, ethanolamine was added to the final reaction mixture to bind any available GLU. This reaction was allowed to continue for 30 minutes in the dark. The final solution was centrifuged at 2000 rpm for 10 minutes, dialyzed twice for 6 hours in a 100-fold excess of saline and three times in the same excess of Hepes buffered saline, and the resulting TRF-GLU-DOX conjugates were ready for use. Biochemical Characterization of the Conjugates:

By using HPLC and polyacrylamide gel electrophoresis as described in (39), the homogeneity of TRF-GLU-DOX conjugates can be determined. Also, by using spectrophotometry, the molecular ratio of DOX-to-TRF can be determined. These techniques repeatedly have revealed a consistent homogeneity of the TRF-GLU-DOX conjugates. In addition, chromatography is not required in the preparation of these conjugates, because there are no aggregates or fragments. This allows for the preparation of large volumes of homogeneous transferrin-drug conjugates, which increases yields and decreases costs.

The expenses caused by losses of TRF and DOX in other types of transferrin-drug conjugates have been an impediment to their use. For example, yields of DOX and TRF are decreased by using procedures such as thiolation that alter the drug and/or protein. Yields also are decreased by using solvent systems and by chromatography used to prepare acid-stable and acid-labile linkages. The GLU bond between DOX and TRF is acid-stable, and yields of useful conjugates prepared according to this invention are high. Indeed, compared to other procedures, the yield for useful conjugate is increased 5-fold.

None of the previously known approaches to the preparation of transferrin-doxorubicin conjugates are capable of producing large amounts of homogeneous conjugates with predetermined ratios of the number of drug molecules per molecule of transferrin. In addition, the other approaches employ chromatography to eliminate aggregates and to harvest fractions that are enriched in homogeneous conjugates. These procedures decrease yields, increase costs, and lack the ability to predetermine molecular ratios.

Another procedure is to mix one milliliter or transferrin (0.5 mm) with one milliliter of doxorubicin (8.5 mM) in 150 mM sodium chloride for 4 minutes, and then add one milliliter of 21.5 mM glutaraldehyde in 150 mM sodium chloride and mix 4 minutes. The preceding reaction is a coupling procedure, which is stopped by the addition of 0.8 milliliters of 37.2 mM ethanolamine in 150 mM sodium chloride and 10 mM Hepes buffer (pH 8) and vortexed for 4 minutes. The mixture then is transferred to dialysis tubing (molecular weight cutoff of 12,000-14,000) dialyzed against 0.5 liters of Hepes-buffered saline in the dark at 5° C. for 3 hours. The dialysis should be repeated at least once with fresh Hepes-buffered saline. The mixture then is centrifuged at 1600 g for 10-minutes at 4° C. and the supernatant is chromatographed at a flow rate of 22 milliliters per hour on a 2.6×34 cm column of Sepharose CL-4B, previously equilibrated in Hepes-buffered saline and calibrated at 5° C. with blue dextran, transferrin and cytochrome C. Elution from the column is monitored at 280 nm, and 3.8 milliliter fractions are collected. The concentration of transferrin and doxorubicin in each fraction is calculated by successive approximation from standard curves for transferrin and doxorubicin, determined by using 280 nm for transferrin and 295 nm for doxorubicin. With modifications, this coupling procedure can be used to prepare transferrin conjugates of other drugs, such as transferrin conjugates of methotrexate and/or antiretroviral drugs. Suitable antiretroviral drugs include but are not limited to nucleoside analogs, or nucleoside reverse transcriptase inhibitors (NRTIs), such as didanosine (ddI, Videx), lamivudine (3TC, Epivir), stavudine (d4T, Zerit), zalcitabine (ddC, Hivid), and zidovudine (AZT, Retrovir), non-nucleoside reverse transcriptase inhibitors (NNRTIs), such as delavirdine (Rescriptor), loviride, and nevirapine (Viramune), and Protease inhibitors, such as indinavir (Crixivan), nelfinavir (Viracept), ritonavir (Norvir) and saquinavir (Invirase).

When pure drug-protein conjugates are isolated, they can be characterized by polyacrylamide gel electrophoresis to determine their molecular weight, and the number of drug molecules per protein molecule can be determined. Experience with drug-protein conjugates in other systems has shown that a functional drug-protein ratio is 0.1-4.0 molecules of drug per molecule of protein (Berczi et al., Arch Biochern Biophy 1993; 300: 356). After conjugation, important steps in the characterization of the conjugates are to (a) determine if the conjugate binds to receptors on the surface of infected cells and not uninfected cells, and (b) determine if the conjugate kills infected cells and not uninfected cells. The binding studies can be done by using flow cytometry, and the killing studies can be done by using microculture techniques to determine the concentration of free drug required to kill 50% of a culture of infected cells compared to the concentration of drug in the drug-protein conjugate required to kill the same number of infected cells. Experience with drug-protein conjugates in other systems indicates that approximately 10-fold more free drug compared to the drug in drug-protein conjugates should be required to kill the same number of infected cells. For a conjugate to be efficacious, it also should kill only a minimum of uninfected cells.

While the present invention has been described in relation to transferrin being the delivery protein, it is known that other proteins exist in the body which are capable of binding to receptor sites on infected cells. If the receptor site is activated in infected cells, and is inactive in uninfected cells, then any protein or other compound which binds to such a receptor site can be used to deliver the drugs used in the present invention. One example of such a binding protein is transcobalamin, which delivers vitamins, especially vitamin B12, to transcobalamin receptors on cells in the human body (Seetheram, Ann Rev Nutr 1999; 19:173). Other examples include but are not limited to somatostatin, epidermal growth factor-like molecules and nonprotein targeting agents such as folic acid-like molecules.

After the drug-protein conjugate has been prepared, it can be purified, characterized and validated for cellular binding and killing properties, and, when the binding and killing experiments show that the conjugate binds to and kills infected cells but not uninfected cells, the conjugate can be aliquoted and sterilized. The sterilization process can be done by exposure to irradiation, such as by using a cesium irradiator, or it can be done by using Millipore filtration techniques.

According to a further aspect of the present invention, there is provided a reagent kit for the treatment of tumors, comprising iron-bearing transferrin and a conjugate of transferrin with an anti-viral agent. The patient's normal cells which have transferrin receptors may be protected against the effects of the conjugate by saturating these receptors with the iron-bearing transferrin before administration of the anti-viral agent.

The present invention also provides a process for determining the susceptibility of tumor cells to anti-viral agents, comprising administering separately to portions of infected cells, conjugates of transferrin with a number of different anti-viral agents. A reagent kit comprising a number of such different conjugates may be provided for this purpose.

The present invention also provides a composition where the conjugate is included in a “cocktail” which also includes at least one free (nonconjugated) anti-viral agent and a method for using the composition.

The conjugates according to the present invention are administered to an animal in an effective amount. In treating viral infections, an effective amount includes an amount effective to reduce the viral titer. The dosage for the conjugates can be determined taking into account the age, weight and condition of the patient and the pharmacokinetics of the anti-viral agent. The amount of the conjugate required for effective treatment will be less than the amount required using the anti-viral agent alone and depends upon the anti-viral agent used. For example, the dosage of a conjugate of transferrin-doxorubicin is expected to be between 0.5-50 mg per 28 day period for a 150 pound (68 kg) person. The dosage can be administered as smaller doses at varying intervals during the 28 day period.

The pharmaceutical compositions of the invention can be administered by a number of routes, including but not limited to orally, topically, rectally, ocularly, vaginally, by pulmonary route, for instance, by use of an aerosol, or parenterally, including but not limited to intramuscularly, subcutaneously, intraperitoneally, intraarterially or intravenously. The compositions can be administered alone, or can be combined with a pharmaceutically-acceptable carrier or excipient according to standard pharmaceutical practice. For the oral mode of administration, the compositions can be used in the form of tablets, capsules, lozenges, troches, powders, syrups, elixirs, aqueous solutions and suspensions, and the like. For parenteral administration, sterile solutions of the conjugate are usually prepared, and the pHs of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. For pulmonary administration, diluents and/or carriers will be selected to be appropriate to allow the formation of an aerosol. It is preferred that the conjugate of the present invention be administered parenterally, i.e. intravenously or intraperitoneally, by infusion or injection.

Preferred embodiments of the present invention are described below. It will be apparent to those of ordinary skill in the art after reading the following description that modifications and variations are possible, all of which are intended to fall within the scope of the claims.

### EXAMPLE 1

Preparation of Homogeneous Conjugates

The synthesis of large amounts of homogeneous transferrin-doxorubicin conjugates with predetermined molecular ratios was done stoichiometrically by employing the only amino group of doxorubicin (DOX), which is at the 3′ amino position, to react with one of the two reactive groups on glutaraldehyde (GLU). The first step was to add GLU drop-wise to DMSO in an ice cold water bath. Next was the drop-wise addition of a saline solution of DOX into a saline solution of GLU+DMSO to a final concentration of a 1:1 molar ratio of DOX-to-GLU. The resulting solution of DOX-GLU was stirred three hours at room temperature in the dark.

The molarities of DOX and GLU were the same in the above reaction in order to produce a final solution of DOX-GLU that contains neither free DOX nor free GLU. However, there is the possibility of free GLU in solution if one GLU reacts with two DOX to produce DOX-GLU-DOX, but this possibility is minimized by the mass action kinetics generated by drop-wise addition of monovalent DOX into the solution of bivalent GLU. The volumes of these reactants are not restricted, so large amounts of homogeneous DOX-GLU can be prepared.

The second step in the conjugation reaction was drop-wise addition of DOX-GLU into a saline solution of transferrin (TRF). The TRF can be either iron-free (apo-transferrin) or iron-saturated (holo-transferrin). The desired molar ratio of DOX to TRF was obtained by appropriately adjusting the volume of TRF. The resulting solution of TRF-GLU-DOX was stirred for 20 hours at room temperature in the dark. Unlike the reaction of DOX with GLU, the reaction of DOX-GLU with TRF is not restricted to one binding site, for the GLU component of DOX-GLU can react with any one of several epsilon-amino lysine groups in the TRF molecule.

The number of DOX molecules bound to TRF was determined in the second step. For example, if the starting ratio of DOX-GLU to TRF was 7.2: 1.0, the final solution of TRF-GLU-DOX would have contained 2.5 molecules of DOX per molecule of TRF. However, if the starting ratio of DOX-GLU to TRF was 4.0:1.0, the final solution of TRF-GLU-DOX would have contained 1.4 molecules of DOX per molecule of TRF. Similarly, if the starting ratio of DOX-GLU to TRF was 2.5:1.0, the final solution of TRF-GLU-DOX would have contained 0.9 molecules of DOX per molecule of TRF. In this way, large amounts of TRF-GLU-DOX with predetermined ratios of DOX-to-TRF can be provided according to the need.

Further steps in the conjugation reaction were the addition of ethanolamine, followed by centrifugation and dialysis. Although reactions with DOX and TRF theoretically consume all of the GLU, ethanolamine was added to the final reaction mixture to bind any available GLU. This reaction was allowed to continue for 30 minutes in the dark. The final solution was centrifuged at 2000 rpm for 10 minutes, dialyzed twice for 6 hours in a 100-fold excess of saline and three times in the same excess of Hepes buffered saline, and the resulting TRF-GLU-DOX conjugates were ready for use. Biochemical Characterization of the Conjugates:

By using HPLC and polyacrylamide gel electrophoresis, the homogeneity of TRF-GLU-DOX conjugates can be determined. Also, by using spectrophotometry, the molecular ratio of DOX-to-TRF can be determined. These techniques repeatedly have revealed a consistent homogeneity of the TRF-GLU-DOX conjugates. In addition, chromatography is not required in the preparation of these conjugates, because there are no aggregates or fragments. This allows for the preparation of large volumes of homogeneous transferrin-drug conjugates, which increases yields and decreases costs;

The expenses caused by losses of TRF and DOX in other types of transferrin-drug conjugates have been an impediment to their use. For example, yields of DOX and TRF are decreased by using procedures such as thiolation that alter the drug and/or protein. Yields also are decreased by using solvent systems and by chromatography used to prepare acid-stable and acid-labile linkages. The GLU bond between DOX and TRF is acid-stable, and yields of DOX and TRF in TRF-DOX conjugates prepared according to this invention are high. Indeed, compared to other known procedures, the yield for TRF is nearly doubled (90% vs 50%), and the yield for DOX is increased 5-fold.

None of the previously known approaches to the preparation of transferrin-doxorubicin conjugates are capable of producing large amounts of homogeneous conjugates with predetermined ratios of the number of drug molecules per molecule of transferrin. In addition, the other approaches employ chromatography to eliminate aggregates and to harvest fractions that are enriched in homogeneous conjugates. These procedures decrease yields, increase costs, and lack the ability to predetermine molecular ratios.

### EXAMPLE 2

Anti-Viral Activity

The effectiveness against different viruses was studied. These were Cytomegalovirus (CMV), Hepatitis B virus (HBV) and HIV. The results versus HIV were particularly good.

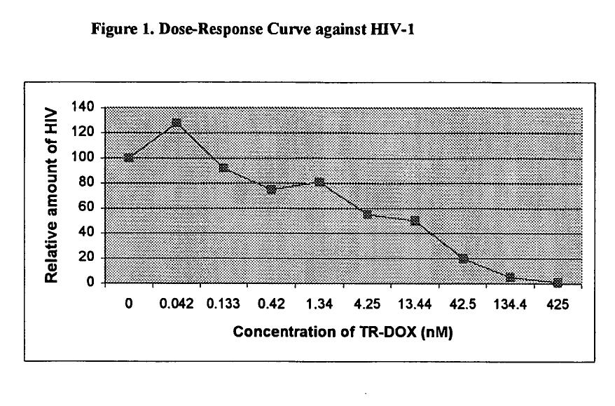

For example, the dose-response curve obtained for the inhibition of the ROJO strain of HIV-1 virus living in human blood cells by TR-DOX is shown in FIG. 1. In this laboratory test system, the TR-DOX clearly had a powerful effect on the AIDS virus at concentrations which suggest that TR-DOX could be used as an effective drug to treat HIV in human AIDS patients.

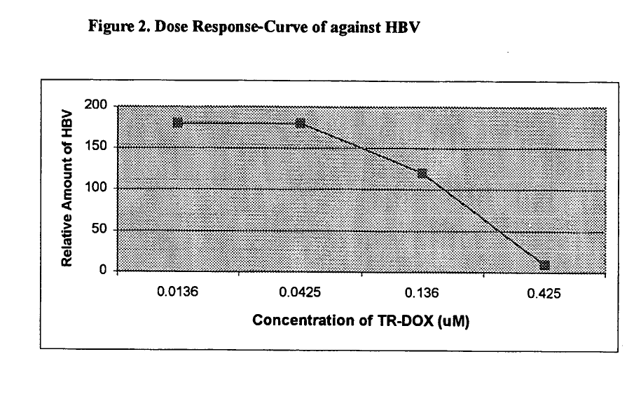

Similarly, FIG. 2 shows the dose-response curve obtained by exposing human liver cells infected with Hepatitis B virus (HBV) to increasing concentrations of TR-DOX. Once again, very low concentrations of TR-DOX were found to cause an almost complete inhibition of the HBV.

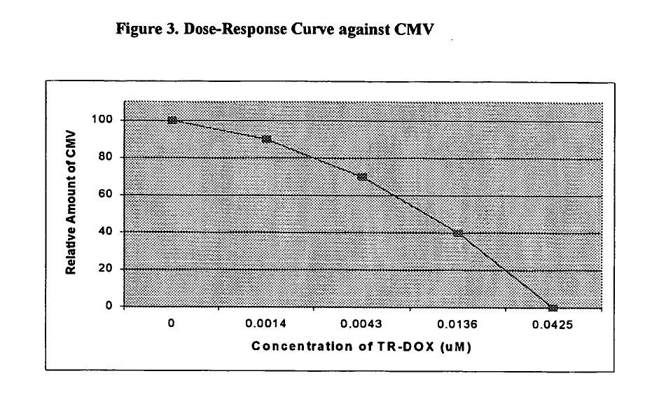

Finally, the effect of TR-DOX on Cytomegalovirus (CMV) living in human lung cells was studied, and once again the data revealed a potent effect of TR-DOX against CMV. The dose-response curve for TR-DOX against CMV is shown below in FIG. 3.

When the relative potency of TR-DOX against CMV was directly compared with the potency of Ganciclovir (which is a widely used anti-viral compound), it was found that TR-DOX was over 200-fold more potent than Ganciclovir in terms of the concentration required to inhibit CMV by 50%.

1. A method for treating a cell infected with a virus, comprising
administering to said cell an anti-viral effective amount of a conjugate containing transferrin and an anti-viral drug, wherein said anti-viral drug is selected from the group consisting of an apoptosis inducing compound, a compound which inhibits the replication of the virus, a cytotoxic antibiotic, an alkylating agent, a plant toxin, and a bacterial mutant toxin.

2. The method according to claim 1, wherein said antiviral agent is selected from the group consisting of doxorubicin, methotrexate, nucleoside reverse transcriptase inhibitors, non-nucleoside reverse transcriptase inhibitors, protease inhibitors, ricin and modified diptheria toxin.

3. The method according to claim 2, wherein said virus is selected from the group consisting of human immunodeficiency virus, cytomegalovirus, and hepatitis virus.

4. The method according to claim 3, wherein said virus is human immunodeficiency virus.

5. A method for treating a patient infected with a virus, comprising administering to said patient a conjugate containing a targeting agent and an anti-viral agent, wherein said anti-viral agent is selected from the group consisting of an apoptosis inducing compound, a compound which inhibits viral reproduction, a cytotoxic antibiotic, an alkylating agent, a plant toxin, and a bacterial mutant toxin and said targeting agent is selected from the group consisting of transferrin and transcobalamin.

6. The method according to claim 5, further comprising administering iron bound transferrin prior to administering said conjugate.

7. The method according to claim 5, wherein said antiviral agent is selected from the group consisting of doxorubicin, methotrexate, nucleoside reverse transcriptase inhibitors, non-nucleoside reverse transcriptase inhibitors and protease inhibitors.

8. The method according to claim 5, wherein said virus is selected from the group consisting of human immunodeficiency virus, cytomegalovirus, and hepatitis virus.

9. The method according to claim 8, wherein said virus is human immunodeficiency virus.

10. The method according to claim 5, further comprising administering a free antiviral agent.

11. A composition comprising a homogeneous conjugate and a carrier, wherein said conjugate contains a viral infected cell targeting agent and an anti-viral agent, and wherein said anti-viral agent is selected from the group consisting of a compound which inhibits viral replication, an alkylating agent, a plant toxin, and a bacterial mutant toxin, and said targeting agent is selected from the group consisting of transferrin and transcobalamin.

12. The composition according to claim 11, further comprising a free antiviral agent.

13. The composition according to claim 11, wherein said targeting agent is transferrin.

14. The composition according to claim 11, wherein said antiviral agent is selected from the group consisting of nucleoside reverse transcriptase inhibitors, non-nucleoside reverse transcriptase inhibitors and protease inhibitors.

15. A composition comprising a homogeneous conjugate, a nonconjugated antiviral agent and a carrier, wherein said conjugate contains a viral infected cell targeting agent and an anti-viral agent, and wherein said anti-viral agent is an apoptosis inducing compound and said targeting agent is selected from the group consisting of transferrin and transcobalamin.

16. A conjugate comprising a targeting agent and an anti-viral agent, wherein said anti-viral agent is a compound which inhibits viral replication, and said targeting agent is selected from the group consisting of transferrin and transcobalamin.

17. The conjugate according to claim 16, wherein said targeting agent is transferrin.

18. The conjugate according to claim 16, wherein said antiviral agent is selected from the group consisting of nucleoside reverse transcriptase inhibitors, non-nucleoside reverse transcriptase inhibitors and protease inhibitors.

19. A method for making a conjugate having a predetermined drug: protein ratio, comprising
a) adding a solution of a drug dropwise to a molar excess of a linker molecule solution to link each drug molecule to one linker molecule in a drug/linker combination; and
b) adding the drug/linker combination to a protein to produce a predetermined drug: protein ratio,
wherein said drug is an antiviral agent.

20. The method according to claim 19, further comprising scavenging any excess linker.

21. The method according to claim 19, wherein said linker is selected from the group consisting of glutaraldehyde, benzoyl hydrazone, maleinimide and N-hydroxysuccinimide.

22. A reagent kit for determining the susceptibility of infected cells to anti-viral agents, comprising two or more conjugates each containing a protein targeting agent and an anti-viral drug, wherein said anti-viral drug is selected from the group consisting of an apoptosis inducing compound, a compound which inhibits the replication of the virus, a cytotoxic antibiotic, an alkylating agent, a plant toxin, and a bacterial mutant toxin and wherein said conjugates have different anti-viral drugs.
